(19) United States
(12) Patent Application Publication
(10) Pub. No.: US 2026/0258331 A1
(43) Pub. Date: Sep. 3, 2026

(54) MODULARLY SCALABLE PHOTOBIOREACTOR

(71) Applicant: Fraunhofer-Gesellschaft zur Förderung der angewandten Forschung e.V., München (DE)

(72) Inventor: Gordon Brinitzer, Stuttgart (DE)

(21) Appl. No.: 18/875,057

(22) PCT Filed: Jun. 13, 2023
(86) PCT No.: PCT/EP2023/065707

§ 371 Date: Dec. 13, 2024

(30) Foreign Application Priority Data
Jun. 14, 2022 (DE) 10 2022 206 019.9

(51) Int. Cl. C12M 1/00; C12M 3/00

(52) U.S. Cl. CPC C12M 21/02; C12M 23/44; C12M 23/58; C12M 31/10

(57) ABSTRACT

The invention pertains to a modular photobioreactor, in particular an artificially illuminated photobioreactor, which is suitable for use in an industrial production environment with a high degree of automation, and to a method for cultivating phototrophic microorganisms.

The present invention relates to a modular photobioreactor, in particular an artificially illuminated modular and scalable photobioreactor, which is suitable for the cultivation of phototrophic microorganisms in an industrial production environment with a high degree of automation. The photobioreactors according to the invention enable maximum biomass and product productivity over a long period of time with optimum biomass quality and low investment, maintenance and operating costs.

A major disadvantage of industrial flat-plate systems is still that the reactor chambers of flat-plate reactors cannot be physically designed to be as wide and high as required, as otherwise the manufacturing costs would rise sharply. In order to scale up the reactor volume to an industrial scale, it is therefore necessary from an economic point of view to arrange many individual flat plates in a system network. Several reactors can be connected in series to form a modular system, for example to scale up from a pre-culture to the desired production volume as quickly as possible or to optimally coordinate multi-stage processes. The individual chambers of such reactors are usually interconnected via external pipes to form a total volume. Due to their design, however, the chambers are often not optimally mixed in order to balance out the concentration gradients that form during cultivation. It may therefore be necessary to monitor all individual volumes independently of each other in order to provide the organism with the best possible growth conditions. The reason for the formation of concentration gradients and inhomogeneous distributions of the cultivated microorganisms within the reactor lies not only in insufficient mixing but also in the dynamics and natural variance of biological systems, in which even the smallest fluctuations in the supply of light or nutrients can lead to differences in terms of growth and/or product formation.

A particular risk in the scaling of flat panel systems is further that if a too large production volume is selected, the entire batch may be lost in the event of contamination. Precise monitoring means a great effort for the system operator and a high investment requirement for the online and offline measurement technology to be used.

Another problem is the enormous amount of space required by photobioreactors that use natural sunlight to cultivate phototrophic microorganisms. To reduce the required footprint, light energy from artificial sources can be used. Short set-up times during the maintenance cycles of such systems are also a decisive factor here for good scalability of large plant systems. The design of the reactor system in accordance with GMP regulations, such as dead space avoidance, residual discharge and sterilizability, must also be solved constructively, for example in order to certify the system and to meet the requirements of an industrial production environment.

When cultivating phototrophic microorganisms, providing the organisms with sufficient light of the appropriate quality is particularly important for the performance of a photobioreactor system. Light is introduced into a cultivation volume via the reactor surface. Depending on the biomass concentration and intensity on the surface, a characteristic absorption profile of the irradiated photons is formed over the layer depth of the volume. In the case of very high intensities, the profile is divided into three zones. At the surface, the cells are exposed to high photon flux densities and are light-inhibited. In the middle zone, the intensity corresponds to moderate conditions and the organisms show the highest photosynthesis rate, while light limitation occurs with increasing mutual shading of the cells in the depth of the reactor. If the light, the biomass concentration or the geometry of the reactor volume is varied, these zones shift accordingly. At very high cell concentrations, the light is already completely absorbed in the first few millimetres; at low cell concentrations, transmission through the cultivation volume can also occur. This relationship characterizes the achievable photosynthesis rate and consequently the performance of the entire reactor. As the reactor is not a static system, the cells move between the zones described. Depending on the mixing strategy used, the cells move statistically along certain trajectories through the reactor volume and are supplied with a light pulse at the surface at a certain frequency before returning to the shaded areas of the reactor. The faster this process takes place, the more cells are statistically supplied with light. A cell experiences a frequency-based light history that is characteristic of the system and kinetic light integration takes place across all cells. A change in the fluid mechanics statistically leads to characteristic light/dark cycles in the volume, whereby these represent a measure of the performance of a reactor. This results in two limiting cases which, depending on the organism used, can lead to good growth in both the one and the other configuration of the reactor system. On the one hand, a reactor with a greater layer thickness and a highly structured geometry can be mixed either by pumping or gassing in order to guide the cells through the reactor by turbulence on specific light/dark trajectories. In this way, a high biomass concentration can be achieved. On the other hand, the layer thickness of the reactor can be greatly reduced with an extreme surface/volume (O/V) ratio. The aim of such a system is to carry out the process under low-light conditions with as little cell shading as possible during the light supply. In this case, the light energy must be diluted accordingly over the entire surface.

This places high demands on photobioreactors that can be used for the efficient cultivation of phototrophic microorganisms on an industrial scale.

The present invention is based on the technical problem of overcoming the disadvantages of the photobioreactors described in the prior art and meeting the high demands placed on photobioreactors for the efficient cultivation of phototrophic microorganisms on an industrial scale. The present invention solves the underlying technical problem by the subject-matter of the independent claims.

The invention relates in particular to a modular photobioreactor comprising a first end piece, a second end piece and at least one reactor compartment arranged between the first end piece and the second end piece, which is characterized in that the photobioreactor comprises at least two cultivation chambers, wherein the at least two cultivation chambers are formed by form-fit joining of the two end pieces and the at least one reactor compartment, wherein the individual cultivation chambers of the photobioreactor are in fluid communication with one another and wherein the two end pieces and the at least one reactor compartment each comprise at least one light source.

The invention relates in particular to a photobioreactor of modular construction, comprising a first end piece, a second end piece and at least one reactor compartment arranged between the first end piece and the second end piece, wherein the photobioreactor comprises at least two cultivation chambers, wherein the at least two cultivation chambers are formed by form-fit joining of the two end pieces and the at least one reactor compartment, wherein the individual cultivation chambers of the photobioreactor are in fluid communication with one another and wherein the two end pieces and the at least one reactor compartment each comprise at least one light source, characterized in that the at least one reactor compartment has two shell elements which are connected to one another in a back-to-back arrangement.

The modular and expandable structure of the photobioreactor according to the invention allows the number of reactor compartments arranged between the end pieces and thus the number of cultivation chambers of the photobioreactor to be increased or reduced as required. The presence of at least one light source, in particular at least one artificial light source, at the two end pieces and the at least one reactor compartment allows the phototrophic microorganisms in the cultivation chambers to be supplied with light energy uniformly and independently of daylight, a compact and space-saving design of the reactor and the modular scalability of the photobioreactor without loss of performance of the reactor due to shading of the cultivated cells and thus a reduction in their photosynthetic performance with an increasing number of reactor compartments arranged next to each other.

In a preferred embodiment of the present invention, a first cultivation chamber is formed by form-fit joining the first end piece to the at least one reactor compartment and a second cultivation chamber is formed by form-fit joining the second end piece to the at least one reactor compartment.

In a particularly preferred embodiment of the present invention, the modular photobioreactor comprises a first end piece, a second end piece and at least two reactor compartments arranged between the first end piece and the second end piece, wherein the photobioreactor comprises at least three cultivation chambers, wherein the at least three cultivation chambers are formed by form-fit joining of the two end pieces and the at least two reactor compartments, wherein the individual cultivation chambers of the photobioreactor are in fluid communication with one another and wherein the two end pieces and the at least two reactor compartments each comprise at least one light source.

According to a preferred embodiment of the present invention, a first cultivation chamber is formed by form-fit joining the first end piece to a first reactor compartment, a second cultivation chamber is formed by form-fit joining the first reactor compartment to the second reactor compartment, and a third cultivation chamber is formed by form-fit joining the second reactor compartment to the second end piece.

Preferably, the photobioreactor according to the invention has at least 2, preferably at least 3, preferably at least 4, preferably at least 5, preferably at least 6, preferably at least 7, preferably at least 8, preferably at least 9, preferably at least 10, preferably at least 15, preferably at least 20, preferably at least 25, reactor compartments arranged between the first end piece and the second end piece.

According to the invention, it can also be provided that the photobioreactor has at most 50, preferably at most 45, preferably at most 40, preferably at most 35, preferably at most 30, preferably at most 25, preferably at most 20, preferably at most 15, preferably at most 10, preferably at most 9, preferably at most 8, preferably at most 7, preferably at most 6, preferably at most 5, preferably at most 4, preferably at most 3, preferably at most 2, reactor compartments arranged between the first end piece and the second end piece.

In a preferred embodiment of the present invention, the photobioreactor comprises at least 3, preferably at least 4, preferably at least 5, preferably at least 6, preferably at least 7, preferably at least 8, preferably at least 9, preferably at least 10, preferably at least 15, preferably at least 20, preferably at least 25, cultivation chambers.

According to a further preferred embodiment of the present invention, the photobioreactor comprises at most 50, preferably at most 45, preferably at most 40, preferably at most 35, preferably at most 30, preferably at most 25, preferably at most 20, preferably at most 15, preferably at most 10, preferably at most 9, preferably at most 8, preferably at most 7, preferably at most 6, preferably at most 5, preferably at most 4, preferably at most 3, cultivation chambers. Preferably, the photobioreactor according to the invention has a first end piece, a second end piece, n reactor compartments arranged between the first end piece and the second end piece and n+1 cultivation chambers, where n≥1, preferably n≥2, preferably n≥3, preferably n≥4, preferably n≥5, preferably n≥6, preferably n≥7, preferably n≥8, preferably n≥9, preferably n≥10, preferably n≥15, preferably n≥20, preferably n≥25.

In a further preferred embodiment of the present invention, the photobioreactor according to the invention has a first end piece, a second end piece, n reactor compartments arranged between the first end piece and the second end piece and n+1 cultivation chambers, where n≤50, preferably n≤45, preferably n≤40, preferably n≤35, preferably n≤30, preferably n≤25, preferably n≤20, preferably n≤15, preferably n≤14, preferably n≤13, preferably n≤12, preferably n≤11, preferably n≤10, preferably n≤9, preferably n≤8, preferably n≤7, preferably n≤6, preferably n≤5.

In a particularly preferred embodiment of the present invention, the modular photobioreactor comprises a first end piece, a second end piece and n reactor compartments arranged between the first end piece and the second end piece, wherein the photobioreactor comprises n+1 cultivation chambers, wherein the n+1 cultivation chambers are formed by form-fit joining of the two end pieces and the n reactor compartments, wherein the individual cultivation chambers of the photobioreactor are in fluid communication with one another, wherein the two end pieces and the n reactor compartments each comprise at least one light source, and wherein n≥1, preferably n≥2, preferably ≥3, preferably ≥4, preferably n≥5, preferably ≥6, preferably ≥7, preferably n≥8, preferably ≥9, preferably ≥10, preferably n≥15, preferably n≥20, preferably n≥25.

Accordingly, the present invention relates in particular to a modular photobioreactor comprising a first end piece, a second end piece and at least n reactor compartments arranged between the first end piece and the second end piece, wherein the photobioreactor comprises at least n+1 cultivation chambers, wherein the at least n+1 cultivation chambers are formed by form-fit joining of the two end pieces and the n reactor compartments, wherein the individual cultivation chambers of the photobioreactor are in fluid communication with one another, wherein the two end pieces and the n reactor compartments each comprise at least one light source, and wherein n≥1, preferably ≥2, preferably ≥3, preferably ≥4, preferably n≥5, preferably ≥6, preferably ≥7, preferably n ≥8, preferably ≥9, preferably ≥10, preferably n≥15, preferably n≥20, preferably n≥25.

According to a preferred embodiment, each of the cultivation chambers of the photobioreactor has at least one upflow region, preferably at least two upflow regions, preferably at least three upflow regions, preferably at least four upflow regions.

In a preferred embodiment, each of the cultivation chambers has at least one downflow region, preferably at least two downflow regions, preferably at least three downflow regions, preferably at least four downflow regions.

Preferably, each of the cultivation chambers has at least two upflow regions and at least two downflow regions.

According to a preferred embodiment, the at least one upflow regions of the cultivation chambers is gassed.

In a preferred embodiment, the gassing of the at least one upflow region of the cultivation chambers takes place via a slotted or perforated membrane, in particular via a slotted or perforated silicone membrane. According to the invention, it may also be provided that the gassing of the at least one upflow region of the cultivation chambers takes place via a porous plastic or ceramic element. In a particularly preferred embodiment of the present invention, the gassing of the upflow regions of the cultivation chambers takes place via a common gassing channel which runs in the foot space of the individual reactor compartments.

In a preferred embodiment of the present invention, the at least one upflow region of the cultivation chambers is gassed with a gas mixture, in particular a CO₂/air mixture.

Preferably, the proportion of CO₂ in the gas mixture, in particular in the CO₂/air mixture, is at least 0.5%, preferably at least 1%, preferably at least 2%, preferably at least 3%, preferably at least 4%, preferably at least 5%, preferably at least 6%, preferably at least 7%, preferably at least 8%, preferably at least 9%.

According to a preferred embodiment of the present invention, the proportion of CO₂ in the gas mixture, in particular in the CO₂/air mixture, is at most 10%, preferably at most 9%, preferably at most 8%, preferably at most 7%, preferably at most 6%, preferably at most 5%, preferably at most 4%, preferably at most 5%, preferably at most 4%, preferably at most 3%, preferably at most 2%, preferably at most 1%.

Particularly preferred, the CO₂ proportion of the gas mixture, in particular the CO₂/air mixture, is 0.5 to 10%, preferably 1 to 9%, preferably 2 to 8%, preferably 3 to 7%, preferably 4 to 6%.

According to a preferred embodiment, the gassing rate is at least 0.01 vvm (vol. gas per vol. culture medium per min), preferably at least 0.025 vvm, preferably at least 0.05 vvm, preferably at least 0.1 vvm, preferably at least 0.2 vvm, preferably at least 0.3 vvm, preferably at least 0.4 vvm, preferably at least 0.5 vvm, preferably at least 0.6 vvm, preferably at least 0.7 vvm, preferably at least 0.8 vvm, preferably at least 0.9 vvm, preferably at least 1 vvm.

Preferably, the gassing rate is at most 1 vvm (vol. gas per vol. culture medium per min), preferably at most 0.9 vvm, preferably at most 0.8 vvm, preferably at most 0.7 vvm, preferably at most 0.6 vvm, preferably at most 0.5 vvm, preferably at most 0.4 vvm, preferably at most 0.3 vvm, preferably at most 0.2 vvm, preferably at most 0.1 vvm, preferably at most 0.05 vvm.

In a particularly preferred embodiment of the present invention, the gassing rate is 0.01 to 1 vvm (vol. gas per vol. culture medium per min), preferably 0.025 to 0.8 vvm, preferably 0.05 to 0.6 vvm, preferably 0.1 to 0.5 vvm, preferably 0.15 to 0.3 vvm.

According to a preferred embodiment of the present invention, the gas mixture, in particular the CO₂/air mixture, is returned from the head region of the photobioreactor for renewed gassing of the at least one upflow region of the cultivation chambers. Particularly preferably, the returned gas mixture, especially the CO₂/air mixture, is enriched again with CO₂ prior to renewed gassing.

In a preferred embodiment of the present invention, the at least one downflow region of a cultivation chamber leads into at least one upflow region of at least one adjacent cultivation chamber. Particularly preferably, the downflow region of a cultivation chamber leads via at least one channel into at least one upflow region of at least one adjacent cultivation chamber.

According to a preferred embodiment of the present invention, the at least one channel connecting the downflow region of a cultivation chamber to the at least one upflow region of at least one adjacent cultivation chamber runs below the at least one light source of the reactor compartments.

By linking the at least one downflow region of a cultivation chamber with the at least one upflow region of at least one adjacent cultivation chamber, the cultivation volume is exchanged between the individual cultivation chambers of the photobioreactor.

In a preferred embodiment of the present invention, the modular photobioreactor has no pump, in particular no pump for mixing the cultivation volume. Preferably, the mixing of the cultivation volume takes place without active pumping.

Particularly preferably, the mixing of the cultivation volume is carried out pneumatically, in particular by moving the cultivation volume through the upflow regions and the downflow regions of the photobioreactor, in particular according to the principle of mammoth pump delivery (airlift principle). By eliminating the need for active pumping of the cultivation volume, the photobioreactor according to the invention advantageously also permits the cultivation of shear-sensitive organisms, in particular shear-sensitive phototrophic microorganisms. Furthermore, the pneumatic mixing reduces the operating, maintenance and repair costs of the photobioreactor and thus increases the economic efficiency of the photobioreactor.

The fluid communication between the individual cultivation chambers advantageously enables mixing of the cultivation volume across all cultivation chambers of the photobioreactor. Particularly preferably, the mixing of the cultivation volume in the cultivation chambers of the photobioreactor which are in fluid communication with one another is driven pneumatically, in particular according to the principle of mammoth pump delivery (airlift principle), that is, driven by the introduction of gas into the lower region of the at least one upflow region of a cultivation chamber, which then rises in the form of gas bubbles and leads to a hydrostatic pressure difference, which in turn results in an upwardly directed flow of the cultivation volume. Since the at least one downflow region of a cultivation chamber is now in fluid communication with the at least one upflow region of at least one adjacent cultivation chamber, there is a flow of the cultivation volume from the at least one downflow region of the cultivation chamber into the upflow region of the at least one adjacent cultivation chamber and thus to a mixing of the cultivation volume between the individual cultivation chambers of the photobioreactor. In this way, a uniform supply of cultivated phototrophic microorganisms with light and nutrients is advantageously ensured and the formation of local concentration gradients in certain areas of the photobioreactor is prevented.

According to a preferred embodiment of the present invention, the at least one light source is an LED light, preferably a high-power LED lamp.

In a preferred embodiment of the present invention, the at least one light source comprises warm white LEDs and/or cool white LEDs, in particular equal proportions of warm white LEDs and cool white LEDs. Preferably, the warm white LEDs have a color temperature of 1500 to 4500 K, preferably 1750 to 3500 K, preferably 2000 to 3000 K. Preferably, the cool white LEDs have a color temperature of 4750 to 8000 K, preferably 5000 to 7000 K, preferably 5250 to 6000 K.

In a preferred embodiment of the present invention, the at least one light source comprises warm white LEDs and/or cool white LEDs and additionally blue LEDs and/or red LEDs. For example, according to the invention, it may be provided that the at least one light source comprises warm white LEDs, cool white LEDs and red LEDs. Alternatively, according to the invention, it can also be provided that the at least one light source comprises warm white LEDs, cool white LEDs and blue LEDs. Particularly preferably, the at least one light source comprises warm white LEDs, cool white LEDs, red LEDs and blue LEDs.

Particularly preferred, the warm white LEDs, cold white LEDs, red LEDs and/or blue LEDs are installed on a common carrier, in particular on a common circuit board.

According to a preferred embodiment, the at least one light source comprises warm white LEDs, cold white LEDs, blue LEDs and red LEDs arranged on a common carrier, in particular on a common circuit board, which can preferably be switched separately and together.

According to a preferred embodiment, the at least one light source comprises at least 50%, preferably at least 55%, preferably at least 60%, preferably at least 65%, preferably at least 70%, preferably at least 75%, white LEDs, in particular warm white and cool white LEDs. According to the invention, it may be provided that the ratio of warm white LEDs to cool white LEDs of the at least one light source is 50:50, preferably 55:45, preferably 60:40, preferably 65:35, preferably 70:30, preferably 75:25. Particularly preferably, the at least one light source comprises white LEDs, of which 50 to 80%, preferably 55 to 75%, preferably 60 to 70%, are warm white LEDs and 20 to 50%, preferably 25 to 45%, preferably 30 to 40%, are cool white LEDs.

In a further preferred embodiment, the at least one light source comprises 0 to 25% blue LEDs and 75 to 100% red LEDs. In a further preferred embodiment of the present invention, the ratio of red LEDs to blue LEDs of the at least one light source is at least 1:3, preferably at least 1:4, preferably at least 1:5, preferably at least 1:6.

Particularly preferably, the luminous efficacy of the at least one light source is at least 150 lm/W (lumens per watt), preferably at least 175 lm/W, preferably at least 200 lm/W, preferably at least 210 lm/W, preferably at least 220 lm/W, preferably at least 230 lm/W, preferably at least 240 lm/W, preferably at least 250 lm/W.

According to a preferred embodiment, the irradiance of the at least one light source is at least 50 W/m² (watts per square meter), preferably at least 75 W/m², preferably at least 100 W/m², preferably at least 125 W/m², preferably at least 150 W/m², preferably at least 175 W/m², preferably at least 200 W/m², preferably at least 225 W/m², preferably at least 250 W/m², preferably at least 275 W/m², preferably at least 300 W/m², preferably at least 325 W/m², preferably at least 350 W/m.²

In a preferred embodiment of the present invention, the irradiance of the at least one light source is at most 400 W/m² (watts per square meter), preferably at most 375 W/m², preferably at most 350 W/m², preferably at most 325 W/m², preferably at most 300 W/m², preferably at most 275 W/m², preferably at most 250 W/m², preferably at most 225 W/m², preferably at most 200 W/m².

Particularly preferred, the irradiance of the at least one light source is 50 to 400 W/m², preferably 100 to 350 W/m², preferably 150 to 300 W/m², preferably 175 to 275 W/m², preferably 200 to 250 W/m².

In a preferred embodiment of the present invention, the at least one light source is part of an illumination plane arranged between two shell elements of a reactor compartment.

Preferably, the illumination plane comprising at least one light source and being arranged between two shell elements of a reactor compartment radiates in two opposite directions. Particularly preferred, the illumination plane arranged between two shell elements of a reactor compartment comprises at least two light sources which are arranged back-to-back to each other and radiate in opposite directions. According to the invention, it may be provided that a cooling device is arranged between the at least two light sources of an illumination plane.

According to a preferred embodiment, the illumination plane has an area of at least 0.5 m², preferably at least 0.6 m², preferably at least 0.7 m², preferably at least 0.8 m², preferably at least 0.9 m², preferably at least 1 m².

In a preferred embodiment of the present invention, the at least one light source, in particular the at least one light source of the illumination plane arranged between two shell elements of a reactor compartment, has a distance of at most 5 cm, preferably at most 4.5 cm, preferably at most 4 cm, preferably at most 3.5 cm, preferably at most 3 cm, preferably at most 2.5 cm, preferably at most 2 cm, preferably at most 1.5 cm, preferably at most 1 cm, preferably at most 0.5 cm, from the surface of the cultivation chambers of the photobioreactor.

Preferably, the at least one light source, in particular the at least one light source of the illumination plane arranged between two shell elements of a reactor compartment, has a distance of at least 0.5 cm, preferably at least 0.75 cm, preferably at least 1 cm, preferably at least 1.25 cm, preferably at least 1.5 cm, preferably at least 1.75 cm, preferably at least 2 cm, preferably at least 2.25 cm, preferably at least 2.5 cm, preferably at least 2.75 cm, preferably at least 3 cm, from the surface of the cultivation chambers of the photobioreactor.

Particularly preferably, the at least one light source, in particular the at least one light source of the illumination plane arranged between two shell elements of a reactor compartment, has a distance of 0.5 to 5 cm, 0.75 to 4 cm, preferably 1 to 3.5 cm, preferably 1.25 to 3 cm, preferably 1.5 to 2.5 cm, from the surface of the cultivation chambers of the photobioreactor.

In a preferred embodiment of the present invention, the at least one reactor compartment comprises two shell elements. In a preferred embodiment of the present invention, the at least one reactor compartment consists of two shell elements. Preferably, the two shell elements of the at least one reactor compartment are connected to each other in a back-to-back arrangement. Particularly preferably, an illumination plane comprising the at least one light source of the reactor compartment is arranged between the two shell elements.

In a preferred embodiment, a reactor compartment of the photobioreactor according to the invention has two shell elements which are connected to one another in a back-to-back arrangement, wherein an illumination plane comprising the at least one light source of the reactor compartment is located between the two shell elements.

According to a particularly preferred embodiment of the present invention, the two shell elements of the at least one reactor compartment are half-shells.

Particularly preferably, the two shell elements, in particular the two half-shells, of a reactor compartment are designed to be mirror-symmetrical with respect to one another. According to this preferred embodiment of the present invention, it may be provided that the mirror plane of the reactor compartment lies in the illumination plane arranged between the two shell elements.

In a further embodiment of the present invention, the two shell elements, in particular the two half-shells, of a reactor compartment are not mirror-symmetrical to each other.

According to a preferred embodiment of the present invention, the first end piece comprises a shell element, in particular a half-shell. Particularly preferably, the first end piece comprises a shell element, in particular a half-shell, and at least one light source.

In a further preferred embodiment of the present invention, the second end piece comprises a shell element, in particular a half-shell. Particularly preferably, the second end piece comprises a shell element, in particular a half-shell, and at least one light source.

According to a particularly preferred embodiment of the present invention, the first and second end pieces each comprise a shell element, in particular a half-shell. Particularly preferably, the first and second end pieces each comprise a shell element, in particular a half-shell, and at least one light source.

According to the invention, it may preferably be provided that, in the assembled state of the modular photobioreactor, each of the two shell elements, preferably each of the two half-shells, of a reactor compartment forms a cultivation chamber with a shell element, preferably a half-shell, of a further reactor compartment or a shell element, preferably a half-shell, of an end piece.

In a preferred embodiment of the present invention, the cultivation chambers formed by the shell elements, preferably half-shells, are sealed by means of a circumferential seal.

According to a further preferred embodiment of the present invention, the two end pieces and the at least one reactor compartment arranged between the two end pieces, preferably the two end pieces and the n reactor compartments arranged between the two end pieces, are arranged horizontally next to each other. Particularly preferably, the at least two cultivation chambers, in particular the n+1 cultivation chambers, are arranged horizontally next to each other.

Preferably, the photobioreactor according to the invention comprises, in addition to the two end pieces and the at least one reactor compartment, in particular in addition to the two end pieces and the at least n reactor compartments, two cover plates arranged at the ends. Particularly preferably, the two end pieces and the at least one reactor compartment, in particular the two end pieces and the at least n reactor compartments, are arranged horizontally next to one another and are pressed together via the end cover plates.

In a preferred embodiment, the end pieces and the reactor compartments arranged next to each other between the first and second end pieces are pressed together hydraulically or by screwing, preferably via cover plates arranged at the ends.

By pressing the end pieces and the at least one reactor compartment arranged between the first and second end piece together, a sealed reactor chamber is formed that is separated from the environment. The force exerted on the end pieces, preferably the force exerted on the end pieces by means of the cover plates, corresponds to at least the spring return force of all reactor compartments and the two end pieces.

Preferably, the at least one reactor compartment of the photobioreactor according to the invention has a height of at least 0.25 m, preferably at least 0.5 m, preferably at least 0.75 m, preferably at least 1 m, preferably at least 1.25 m, preferably at least 1.5 m, preferably at least 1.75 m, preferably at least 2 m.

According to a preferred embodiment, the at least one reactor compartment of the photobioreactor according to the invention has a height of at most 5 m, preferably at most 4.5 m, preferably at most 4 m, preferably at most 3.5 m, preferably at most 3 m, preferably at most 2.5 m, preferably at most 2 m, preferably at most 1.5 m.

In a particularly preferred embodiment of the present invention, the at least one reactor compartment has a height of 0.25 to 5 m, preferably 0.5 to 4.5 m, preferably 0.75 to 4 m, preferably 1 to 3.5 m, preferably 1.25 to 3 m, preferably 1.5 to 2.5 m.

Preferably, the first and second end pieces of the photobioreactor according to the invention have a height of at least 0.25 m, preferably at least 0.5 m, preferably at least 0.75 m, preferably at least 1 m, preferably at least 1.25 m, preferably at least 1.5 m, preferably at least 1.75 m, preferably at least 2 m.

According to a preferred embodiment, the first and second end pieces of the photobioreactor according to the invention have a height of at most 5 m, preferably at most 4.5 m, preferably at most 4 m, preferably at most 3.5 m, preferably at most 3 m, preferably at most 2.5 m, preferably at most 2 m, preferably at most 1.5 m.

In a particularly preferred embodiment of the present invention, the first and second end pieces of the photobioreactor according to the invention have a height of 0.25 to 5 m, preferably 0.5 to 4.5 m, preferably 0.75 to 4 m, preferably 1 to 3.5 m, preferably 1.25 to 3 m, preferably 1.5 to 2.5 m.

Preferably, the at least one reactor compartment of the photobioreactor according to the invention has a width of at least 0.25 m, preferably at least 0.5 m, preferably at least 0.75 m, preferably at least 1 m, preferably at least 1.25 m, preferably at least 1.5 m, preferably at least 1.75 m, preferably at least 2 m.

According to a preferred embodiment, the at least one reactor compartment of the photobioreactor according to the invention has a width of at most 5 m, preferably at most 4.5 m, preferably at most 4 m, preferably at most 3.5 m, preferably at most 3 m, preferably at most 2.5 m, preferably at most 2 m, preferably at most 1.5 m.

In a particularly preferred embodiment of the present invention, the at least one reactor compartment has a width of 0.25 to 5 m, preferably 0.5 to 4.5 m, preferably 0.75 to 4 m, preferably 1 to 3.5 m, preferably 1.25 to 3 m, preferably 1.5 to 2.5 m.

Preferably, the first and second end pieces of the photobioreactor according to the invention have a width of at least 0.25 m, preferably at least 0.5 m, preferably at least 0.75 m, preferably at least 1 m, preferably at least 1.25 m, preferably at least 1.5 m, preferably at least 1.75 m, preferably at least 2 m.

According to a preferred embodiment, the first and second end pieces of the photobioreactor according to the invention have a width of at most 5 m, preferably at most 4.5 m, preferably at most 4 m, preferably at most 3.5 m, preferably at most 3 m, preferably at most 2.5 m, preferably at most 2 m, preferably at most 1.5 m.

In a particularly preferred embodiment of the present invention, the first and second end pieces of the photobioreactor according to the invention have a width of 0.25 to 5 m, preferably 0.5 to 4.5 m, preferably 0.75 to 4 m, preferably 1 to 3.5 m, preferably 1.25 to 3 m, preferably 1.5 to 2.5 m.

Particularly preferably, the cultivation chambers, in particular the cultivation chambers formed by the two end pieces and the reactor compartments arranged between the first and second end pieces, have a maximum chamber depth, in particular maximum layer thickness, of at least 0.5 cm, preferably at least 1 cm, preferably at least 1.5 cm, preferably at least 2 cm, preferably at least 2.5 cm, preferably at least 3 cm, preferably at least 3.5 cm, preferably at least 4 cm, preferably at least 4.5 cm, preferably at least 5 cm.

According to a preferred embodiment of the present invention, the cultivation chambers, in particular the cultivation chambers formed by the two end pieces and the reactor compartments arranged between the first and second end pieces, have a maximum chamber depth, in particular maximum layer thickness, of at most 10 cm, preferably at most 9 cm, preferably at most 8 cm, preferably at most 7 cm, preferably at most 6 cm, preferably at most 5 cm, preferably at most 4.5 cm, preferably at most 4 cm, preferably at most 3.5 cm, preferably at most 3 cm, preferably at most 2.5 cm, preferably at most 2 cm, preferably at most 1.5 cm.

Particularly preferably, the cultivation chambers, in particular the cultivation chambers formed by the two end pieces and the reactor compartments arranged between the first and second end pieces, have a non-uniform chamber depth, in particular a non-uniform layer thickness. Particularly preferably, the cultivation chambers, in particular the cultivation chambers formed by the two end pieces and the reactor compartments arranged between the first and second end pieces, have a non-uniform chamber depth, in particular a non-uniform layer thickness, in the range from 0.5 to 10 cm, preferably 0.5 to 7.5 cm, preferably 0.5 to 5 cm, preferably 1 to 4.5 cm, preferably 1 to 4 cm, preferably 1.5 to 3.5 cm, preferably 1.5 to 3 cm.

According to a preferred embodiment of the present invention, the cultivation chambers, in particular the cultivation chambers formed by the two end pieces and the at least one reactor compartment arranged between the first and second end piece, each have a volume of at least 5 liters, preferably at least 10 liters, preferably at least 20 liters, preferably at least 30 liters, preferably at least 40 liters, preferably at least 50 liters, preferably at least 75 liters, preferably at least 100 liters, preferably at least 150 liters, preferably at least 200 liters, preferably at least 250 liters, preferably at least 500 liters, preferably at least 750 liters, preferably at least 1,000 liters, preferably at least 1,500 liters, preferably at least 2,000 liters, preferably at least 2,500 liters, preferably at least 3,000 liters, preferably at least 3,500 liters, preferably at least 4,000 liters, preferably at least 5,000 liters.

In a preferred embodiment, the cultivation chambers, in particular the cultivation chambers formed by the two end pieces and the at least one reactor compartment arranged between the first and second end piece, each have a volume of at most 20,000 liters, preferably at most 15,000 liters, preferably at most 10,000 liters, preferably at most 7,500 liters, preferably at most 5,000 liters, preferably at most 4,000 liters, preferably at most 3,000 liters, preferably at most 2,500 liters, preferably at most 2,000 liters, preferably at most 1,500 liters, preferably at most 1,000 liters, preferably at most 750 liters, preferably at most 500 liters, preferably at most 400 liters, preferably at most 300 liters, preferably at most 200 liters, preferably at most 150 liters, preferably at most 100 liters, preferably at most 75 liters, preferably at most 50 liters.

Particularly preferably, the cultivation chambers, in particular the cultivation chambers formed by the two end pieces and the at least one reactor compartment arranged between the first and second end piece, each have a volume of 5 to 20,000 liters, preferably 5 to 10,000 liters, preferably 10 to 7,500 liters, preferably 10 to 5,000 liters, preferably 20 to 2,500 liters, preferably 20 to 1,000 liters, preferably 30 to 500 liters, preferably 30 to 250 liters.

In a further preferred embodiment, the photobioreactor according to the invention has a total volume of at least 100 liters, preferably at least 150 liters, preferably at least 200 liters, preferably at least 250 liters, preferably at least 300 liters, preferably at least 350 liters, preferably at least 400 liters, preferably at least 450 liters, preferably at least 500 liters, preferably at least 750 liters, preferably at least 1,000 liters, preferably at least 2,500 liters, preferably at least 5,000 liters, preferably at least 7,500 liters, preferably at least 10,000 liters.

In a particularly preferred embodiment of the present invention, the cultivation chambers formed by shell elements, in particular by the half-shells, of the reactor compartments are segmented.

Particularly preferably, the at least one upflow region of the cultivation chambers formed by the shell elements, in particular by the half-shells, of the reactor compartments is segmented.

In a preferred embodiment of the present invention, the reactor compartments, in particular the shell elements, preferably the half-shells, of the reactor compartments, have a horizontal segmentation in the at least one upflow region, in particular a horizontal segmentation in the form of reactor half-shells arranged horizontally one above the other.

Particularly preferred, the horizontal segmentation of the shell elements, preferably the half-shells, of the reactor compartments, in particular the horizontal segmentation in the form of reactor half-shells arranged horizontally one above the other, forms static mixers in the assembled state of the photobioreactor in the at least one upflow region of the cultivation chambers.

Preferably, the cultivation chambers of the photobioreactor, in particular the at least one upflow region of the cultivation chambers, comprise at least 2, preferably at least 3, preferably at least 4, preferably at least 5, preferably at least 6, preferably at least 7, preferably at least 8, preferably at least 9, preferably at least 10, preferably at least 15, preferably at least 20, preferably at least 25, preferably at least 30, preferably at least 35, preferably at least 40, preferably at least 45, preferably at least 50, preferably at least 55, preferably at least 60, static mixers, in particular static mixers arranged one above the other.

According to a preferred embodiment of the present invention, the cultivation chambers of the photobioreactor, in particular the at least one upflow region of the cultivation chambers, have at most 100, preferably at most 90, preferably at most 80, preferably at most 70, preferably at most 60, preferably at most 50, preferably at most 40, preferably at most 35, preferably at most 30, preferably at most 25, preferably at most 20, preferably at most 15, preferably at most 10, preferably at most 9, preferably at most 8, preferably at most 7, preferably at most 6, preferably at most 5, static mixers, in particular static mixers arranged one above the other.

The present invention also relates to a method for cultivating phototrophic microorganisms, comprising the steps of:
a) Providing a culture medium comprising at least one phototrophic microorganism,
b) Providing a modular photobioreactor according to the invention,
c) Cultivating the at least one phototrophic microorganism in the modular photobioreactor according to the invention.

A further aspect of the present invention relates to the use of a modular photobioreactor according to the invention for cultivating phototrophic microorganisms.

The embodiments described in connection with the modular photobioreactor according to the invention apply mutatis mutandis to the method for cultivating phototrophic microorganisms according to the invention and the use of the modular photobioreactor for cultivating phototrophic microorganisms according to the invention.

According to the invention, the term “modular photobioreactor” is understood to mean a bioreactor suitable for the cultivation of phototrophic microorganisms, which is characterized in that the bioreactor is constructed from several components, in particular two end pieces and at least one reactor compartment arranged between the two end pieces, and can be scaled according to the requirements placed on it by removing or adding individual components, in particular individual reactor compartments. In addition to the scalability of the reactor volume, the “modular structure” of the photobioreactor according to the invention also allows the photobioreactor to be easily assembled and disassembled for repair and maintenance purposes. In particular, the “modular photobioreactor” according to the invention is characterized in that it comprises a first and a second end piece, between which individual reactor compartments can be arranged, whereby cultivation chambers are formed by form-fit joining of the two end pieces with the individual reactor compartments arranged therebetween, which are in fluid communication with each other, so that the total volume of the photobioreactor can be scaled by the number of reactor compartments arranged between the two end pieces.

In connection with the present invention, the term “cultivation chamber” is used for a delimited space defined by walls within the modular photobioreactor according to the invention, which has a volume that is suitable for holding culture medium and for cultivating phototrophic microorganisms present in the culture medium. Preferably, the individual “cultivation chambers” of the photobioreactor according to the invention are characterized by the fact that they are formed by form-fit joining of the two end pieces and the at least one reactor compartment. Particularly preferred, the “cultivation chambers” comprise at least one upflow region and at least one downflow region.

The term “reactor compartment” refers to a component of the modular photobioreactor according to the invention which, together with a further “reactor compartment” and/or an end piece of the photobioreactor, forms at least one cultivation chamber by form-fit joining. Particularly preferred, a reactor compartment has two shell elements, in particular two half-shells, which are arranged back-to-back and connected to each other.

According to the invention, the term “upflow region” (also: “riser”) refers to a delimitable region of a cultivation chamber within which the introduced gas and the culture medium in the cultivation chamber are transported vertically upwards.

According to the invention, the term “downflow region” (also: “downcomer”/“downer”) refers to a delimitable region of a cultivation chamber within which the culture medium in the cultivation chamber is transported vertically downwards.

According to the invention, the term “warm white LED” refers to a light-emitting diode which emits visible light with a color temperature in the range from 1500 to 4500 K, preferably 1750 to 3500 K, preferably 2000 to 3000 K, when an electric current flows in the forward direction.

According to the invention, the term “cool white LED” is used for a light-emitting diode which emits visible light with a color temperature in the range from 4750 to 8000 K, preferably 5000 to 7000 K, preferably 5250 to 6000 K, when electric current flows in the forward direction.

In the context of the present invention, the term “blue LED” is understood to mean a light-emitting diode which emits visible light with a wavelength in the range from 400 to 500 nm, preferably 410 to 490 nm, preferably 420 to 480 nm, preferably 430 to 470 nm, when electric current flows in the forward direction.

According to the invention, a “red LED” is understood to be a light-emitting diode which emits visible light with a wavelength in the range from 600 to 700 nm, preferably 610 to 690 nm, preferably 620 to 680 nm, preferably 630 to 670 nm, when an electric current flows in the forward direction.

In the context of the present invention, the terms “comprising” and “containing” are understood to mean that, in addition to the elements explicitly covered by these terms, further elements not explicitly mentioned may be present. In the context of the present invention, these terms are also understood to mean that only the explicitly mentioned elements are included and that no further elements are present. In this particular embodiment, the meaning of the terms “comprising” and “containing” is synonymous with the term “consisting of”.

Furthermore, the terms “comprising” and “containing” also cover compositions which, in addition to the explicitly mentioned elements, also contain other elements which are not mentioned but which are of a functionally and qualitatively subordinate nature. In this embodiment, the terms “comprising” and “containing” are synonymous with the term “essentially consisting of”.

In connection with the present invention, the term “and/or” is understood to mean that all members of a group which are connected by the term “and/or” are disclosed both alternatively to one another and cumulatively to one another in any combination. This means for the expression “A, B and/or C” that the following disclosure content is to be understood: a) A or B or C, or b) (A and B), or c) (A and C), or d) (B and C), or e) (A and B and C).

If, in connection with the present invention, the first and second decimal places or the second decimal place are/is not specified, these are/are to be set as 0.

Further preferred embodiments are shown in the dependent claims.

The invention is illustrated below, without limiting the general idea of the invention, by means of figures.

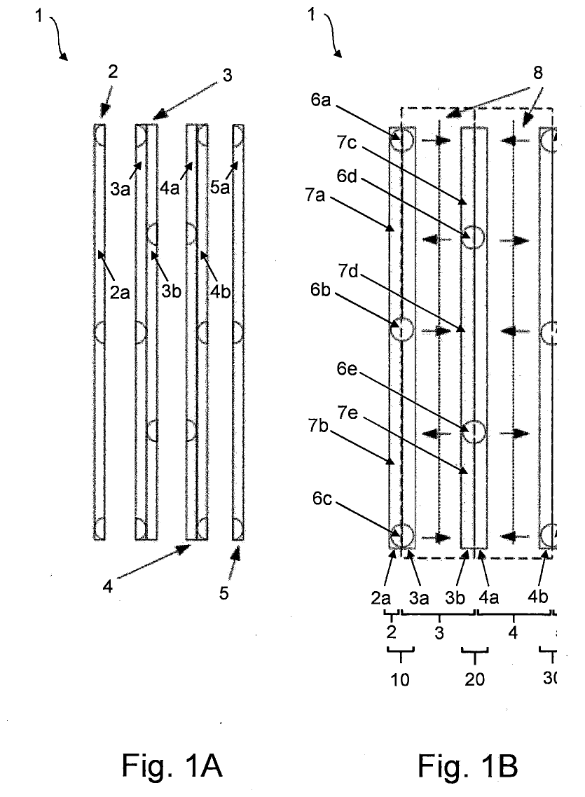

FIG. 1A shows the top view on a modular photobioreactor (1) according to the invention comprising two end pieces (2), (5) and two reactor compartments (3), (4) in the unassembled state. Each of the reactor compartments (3), (4) comprises two shell elements (3a, 3b), (4a, 4b) which are connected to one another in a back-to-back arrangement and can be connected in a form-fit manner with the shell elements (2a), (5a) of the end pieces (2), (5).

FIG. 1B shows the top view on a modular photobioreactor according to the invention in the assembled state. Each of the reactor compartments (3), (4) comprises two shell elements (3a, 3b, 4a, 4b) which are arranged back-to-back. The form-fit joining of the shell element (2a) of the first end piece (2) to the one shell element (3a) of the reactor compartment (3) creates the cultivation chamber (10), which has two upflow regions (7a, 7b) and three downflow regions (6a, 6b, 6c). In the assembled state of the photobioreactor (1), the cultivation chamber (20) comprising three upflow regions (7c, 7d, 7e) and two downflow regions (6d, 6e) is formed between the second shell element (3b) of the reactor compartment (3) and a shell element (4a) of the reactor compartment (4). A further cultivation chamber (30) with two upflow regions (7f, 7g) and three downflow regions (6f, 6g, 6h) is formed by form-fit joining of the second shell element (4b) of the reactor compartment (4) to the shell element (5a) of the second end piece (5). The arrows show the direction of flow of the culture volume from the downflow regions (6a, 6b, 6c, 6d, 6e, 6f, 6g, 6h) of the cultivation chambers (10, 20, 30) of the photobioreactor (1) to the upflow regions (7a, 7b, 7c, 7d, 7e, 7f, 7g) of the respective adjacent cultivation chamber(s). The reactor compartments (3, 4) each have an illumination plane (8) comprising at least one light source arranged between the shell elements (3a, 3b, 4a, 4b).

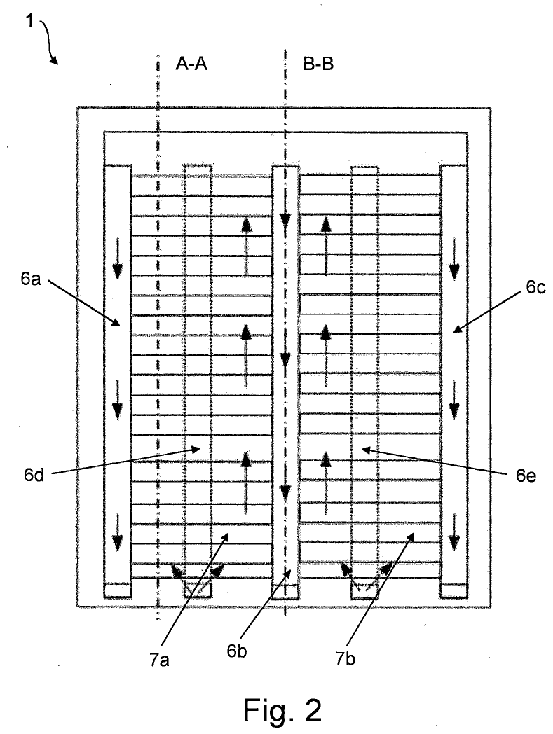

FIG. 2 shows the front view of a modular photobioreactor (1) according to the invention. A first cultivation chamber with three downflow regions (6a, 6b, 6c) and two upflow regions (7a, 7b) is shown in the foreground. In the background, two downflow areas (6d, 6e) of the next cultivation chamber are illustrated in dashed lines. The arrows show the direction of flow of the culture volume. The culture volume flows from the downflow regions (6d, 6e) to the upflow regions (7a, 7b) of the cultivation chamber which is shown in the foreground.

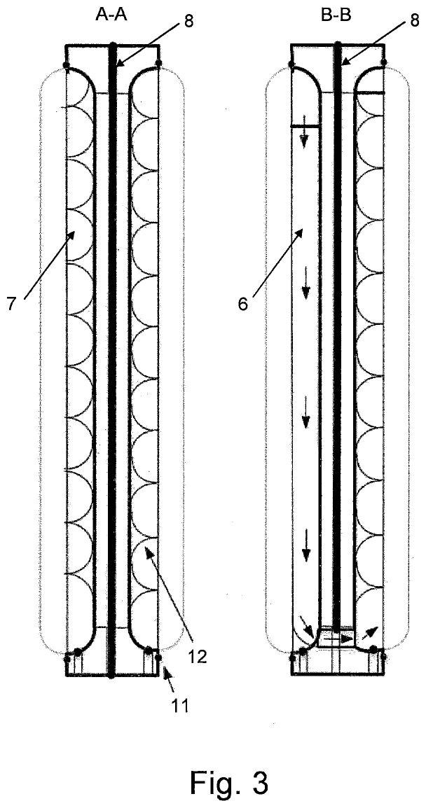

FIG. 3 shows sections of a reactor compartment along the sectional planes A-A and B-B of FIG. 2. It can be seen from the section along the sectional plane A-A (left) that the upflow region (7) of the reactor compartment shown has static mixers (12) arranged one above the other in the form of horizontally extending reactor half-shells. An illumination plane (8) comprising at least one light source is arranged between the two shell elements of the reactor compartment. To seal the cultivation chambers of the photobioreactor, each of the shell elements of a reactor compartment can comprise a circumferential seal (11).

In the section along the sectional plane B-B (right), the downflow region (6) of a reactor compartment is shown. The culture volume is transported vertically downwards through the downflow region (6) of the cultivation chamber, passes under the illumination plane (8), which is arranged between the two shell elements of the reactor compartment shown, and finally flows into the upflow region of the adjacent cultivation chamber.

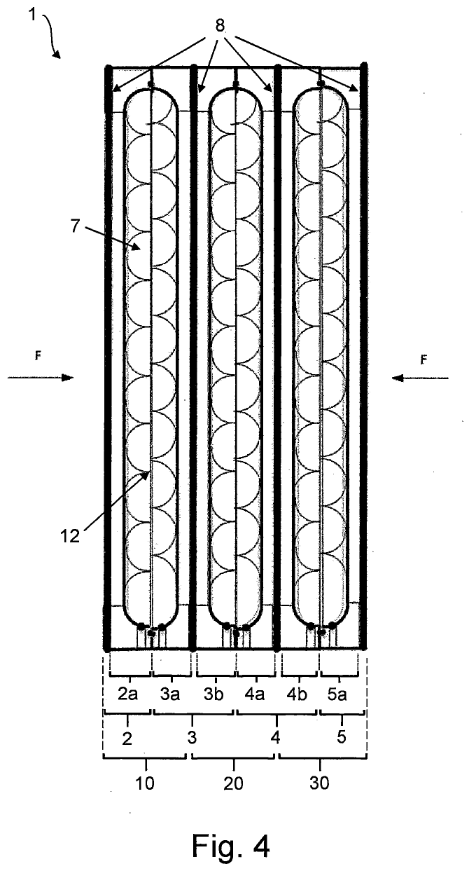

FIG. 4 a section through the upflow regions (7) of three cultivation chambers (10, 20, 30) arranged next to one another, which are formed by form-fit joining of a first end piece (2), two reactor compartments (3, 4) and a second end piece (5). The two end pieces (2, 5) and the two reactor compartments (3, 4) each have an illumination plane (8) comprising at least one light source arranged between the shell elements (3a, 3b, 4a, 4b) of the reactor compartments (3, 4) or on the shell elements (2a, 5a) of the two end pieces (2, 5), respectively. The two end pieces (2, 5) and the two reactor compartments (3, 4) arranged between them of the photobioreactor (1) shown are pressed together by the force F exerted on the two end pieces (2, 5). Disassembly of the modular photobioreactor or rather the installation or removal of individual reactor compartments is possible in a simple manner by releasing the force F exerted on the two end pieces (2, 5).

### LIST OF REFERENCE SIGNS

1 photobioreactor
2 first end piece
2a half-shell of the first end piece
3 first reactor compartment
3a first half-shell of the first reactor compartment
3b second half-shell of the first reactor compartment
4 second reactor compartment
4a first half-shell of the second reactor compartment
4b second half-shell of the second reactor compartment
5 second end piece
5a half-shell of the second end piece
6 downflow region
6a-6h downflow regions
7 upflow region
7a-7g upflow region
8 illumination plane
10 first cultivation chamber
11 seal
12 static mixer
20 second cultivation chamber
30 third cultivation chamber
A-A sectional plane through the upflow region of a reactor compartment
B-B sectional plane through the downflow region of a reactor compartment

1. A modular photobioreactor (1), comprising a first end piece (2), a second end piece (5) and at least one reactor compartment arranged between the first end piece (2) and the second end piece (5), wherein the photobioreactor comprises at least two cultivation chambers, wherein the at least two cultivation chambers are formed by form-fit joining of the two end pieces (2, 5) and the at least one reactor compartment, wherein the individual cultivation chambers of the photobioreactor (1) are in fluid communication with one another and wherein the two end pieces (2, 5) and the at least one reactor compartment each comprise at least one light source, characterized in that the at least one reactor compartment has two shell elements which are connected to one another in a back-to-back arrangement.

2. The modular photobioreactor (1) according to claim 1, characterized in that a first cultivation chamber is formed by form-fit joining of the first end piece (2) to the at least one reactor compartment and a second cultivation chamber is formed by form-fit joining of the second end piece (5) to the at least one reactor compartment.

3. The modular photobioreactor (1) according to claim 1, characterized in that each of the cultivation chambers has at least one downflow region (6) and at least one upflow region (7).

4. The modular photobioreactor (1) according to claim 3, characterized in that the at least one downflow region (6) of a cultivation chamber leads into at least one upflow region (7) of at least one adjacent cultivation chamber.

5. The modular photobioreactor (1) according to claim 1, characterized in that the at least one light source is an LED light.

6. The modular photobioreactor (1) according to claim 1, characterized in that an illumination plane (8) comprising the at least one light source of the reactor compartment is located between the two shell elements.

7. The modular photobioreactor (1) according to claim 1, characterized in that the two end pieces (2, 5) and the at least one reactor compartment are arranged horizontally next to one another and are pressed together via end cover plates.

8. The modular photobioreactor (1) according to claim 1, characterized in that the at least one reactor compartment has a height of 0.5 to 4 m, preferably 1 to 3 m.

9. The modular photobioreactor (1) according to claim 1, characterized in that the at least one reactor compartment has a width of 0.5 to 4 m, preferably 1 to 3 m.

10. A method for cultivating phototrophic microorganisms comprising the steps of:
a) providing a culture medium comprising at least one phototrophic microorganism,
b) providing a modular photobioreactor (1) according to claim 1,
c) cultivating the at least one phototrophic microorganism in the modular photobioreactor (1).

11. Use of a modular photobioreactor (1) according to claim 1 for cultivating phototrophic microorganisms.
